(19) United States
(12) Patent Application Publication
(10) Pub. No.: US 2013/0052192 A1
(43) Pub. Date: Feb. 28, 2013

(54) Activatable Constructs

(71) Applicants: Jais Rose Bjelke, Smoerum (DK); Susanne Nedergaard Grell, Soeborg (DK); Thomas Egebjerg, Ganloese (DK)

(72) Inventors: Jais Rose Bjelke, Smoerum (DK); Susanne Nedergaard Grell, Soeborg (DK); Thomas Egebjerg, Ganloese (DK)

(73) Assignee: Novo Nordisk A/S, Bagsvaerd (DK)

(21) Appl. No.: 13/579,367

(22) PCT Filed: Feb. 18, 2011
(86) PCT No.: PCT/EP11/52416

§ 371 Date: Nov. 8, 2012

Related U.S. Application Data
(60) Provisional application No. 61/307,606, filed on Feb. 24, 2010.

(30) Foreign Application Priority Data
Feb. 19, 2010 (EP) 10154178.7

(51) Int. Cl. A61K 39/395; C12N 1/19; A61P 9/00; C07K 19/00; C12N 15/62; C12N 1/21; C12N 5/10

(52) U.S. Cl. 424/134.1; 435/252.33; 435/254.23; 435/254.21; 435/328; 530/387.3; 536/23.4

(57) ABSTRACT

The current invention relates to a construct, a polynucleotide that encodes said construct and a cell that expresses said construct. Furthermore, the current invention relates to the use of said construct for the treatment of bleedings and their associated co-morbidities and to a method of treatment of bleedings, inflammation and metastasis of cancer cells.

### FIELD OF THE INVENTION

The current invention relates to a construct, a polynucleotide that encodes said construct, a cell that expresses said construct, the use of said construct for the treatment of the co-morbidities associated with the disruption of [a] blood vessel[s] and a method of treatment of coagulopathy, inflammation and metastasis of cancer cells.

### BACKGROUND OF THE INVENTION

Platelets derive from their cellular predecessor, megakaryocytes, in the bone marrow. Normal resting platelets freely flow throughout the blood circulation when the endothelium is intact. When the single-layered endothelial barrier is damaged, resting platelets adhere to sub-endothelial structures by means of glycoprotein (GP) receptors. For example, GPIaIIa and GPVI bind collagen; GPIcIIa binds fibronectin; GPIc*IIa binds laminin and GPIb-V-IX binds von Willebrand Factor (vWF) polymers. Adhesion of platelets in this manner causes them to change shape and release their alpha and dense granules. In turn, this results in the exposure of a plethora of other glycoprotein platelet receptors, such as GPIIbIIIa (which binds fibrinogen/fibrin), TLT-1 and CREB-2; as well as the release of coagulation factors I (fibrinogen), V and XI; other procoagulants such as ADP, Ca²⁺ and serotonin; anti-coagulants such as tissue factor pathway inhibitor (TFPI); and compounds such as platelet derived growth factor (PDGF), essential to platelet replenishment and healing. Activated platelets bind to one other and cross-link fibrin in a rapid reaction. Activated platelets also play a vital role in the homing of leukocytes to sites of vascular injury and inflammation, by means of selectins displayed on their [cell] membranes.

Tissue factor (TF), a transmembrane glycoprotein, is the primary cellular initiator of blood coagulation. It is predominantly expressed on the surface of sub-endothelial cells, such as smooth muscle cells and fibroblasts, and binds both coagulation Factor VII (FVII) and Factor VIIa (FVIIa) when the integrity of the endothelium is interrupted, such as when blood vessels are severed. When TF binds FVII, it promotes FVII to FVIIa activation. TF also greatly enhances the proteolytic activity of Factor VIIa towards its physiologic substrates, Factors IX and X. It does so by providing a scaffold for optimal macro-molecular exosite interaction and by inducing conformational changes in the protease domain of Factor VIIa that results in correct definition of the active-site region. Hence, TF is a co-factor for FVIIa in the initiation complex of what is traditionally referred to as the extrinsic pathway of blood coagulation. Subsequent steps of the coagulation cascade finally result in the formation of a fibrin polymer which is bound by activated platelets and cross-linked with FXIIIa.

Hence, activated platelets and the fibrin polymer product of the coagulation cascade together form a stable blood clot and promote tissue healing.

In subjects with a coagulopathy, such as in human beings with haemophilia A and B, various steps of the coagulation cascade are rendered dysfunctional due to, for example, the absence or insufficient presence of a coagulation factor. Such dysfunction of one part of coagulation results in insufficient blood coagulation and potentially life-threatening bleeding.

One object of the current invention is to provide a construct that is suitable for use as a procoagulant drug in such subjects. A second object of the current invention is to provide a construct that is activated at the desired physical point of initiation of blood coagulation. A third object of the current invention is to provide a construct that upregulates blood coagulation in a physiologically suitable microenvironment. A further object of the current invention is to direct a monoclonal antibody, or a biologically functional fragment or variant thereof, to the surface of activated platelets or endothelial cells. Thus, the object is to enable the initiation of blood coagulation on the surface of activated platelets that are located intravascularly or extravascularly. This is in contrast with the normal and exclusively subendothelial—typically extravascular—initiation of blood coagulation.

It is known that some of the processes/molecules that are involved in haemostasis are intricately intertwined with tissue healing and, thus, also with pathological processes such as inflammation and metastasis of cancer cells. One object of the current invention is thus to provide a construct that may also be used to treat the co-morbidities that arise in connection with vessel rupture in bleeding patients, notably inflammatory diseases; as well as autoimmune diseases and the metastasis of cancer cells.

### SUMMARY OF THE INVENTION

The current invention relates to a construct comprising (i) a ligand such as a monoclonal antibody (mAB), or a fragment thereof, (ii) a cleavable motif (CM) and (iii) a polypeptide sequence that comprises the epitope motif (EM) of (i). Said construct may further comprise tissue factor (TF), or a biologically functional variant or fragment thereof. Said mAB of said construct may be capable of binding a receptor on a cell selected from the group consisting of an activated platelet, an activated endothelial cell and a leukocyte. In one aspect, said mAB is capable of binding a receptor on an activated platelet. This receptor may be a selectin, such as an E-selectin (CD62E), a P-selectin (CD62P) or an L-selectin (CD62L). In one embodiment, said receptor is a P-selectin (CD62P).

The construct may be engineered such that the C-terminus of said CM is covalently linked to the N-terminus of the heavy chain of said monoclonal antibody or fragment thereof. The construct may be engineered such that the C-terminus of said cleavable motif is covalently linked to the N-terminus of the light chain of said monoclonal antibody or fragment thereof. The C-terminus of said epitope motif may be covalently linked to the N-terminus of said cleavable motif.

The CM of the construct of the current invention may be cloven by an enzyme. This enzyme may be a protease selected from the group consisting of thrombin, Factor VIIa (FVIIa), Factor IXa (FIXa), Factor Xa (FXa), Factor XIa (FXIa), Factor XIIa (FXIIa), kallikrein, activated protein C (APC), plasmin, tissue plasminogen activator (tPA) and urokinase-type plasminogen Activator (uPA). The CM may be the cleavage site of a protease. In one specific embodiment, said CM may be fibrinopeptide A (FpA), or a fragment thereof; such as amino acids 40-49 (DFLAEGGGVR) of SEQ ID NO: 3. The EM may comprise an amino acid sequence that corresponds to residues 20-39 (SVLQCLATGNWNSVPPECQA) of SEQ ID NO: 3. The mAB of the construct provided may comprise SEQ ID NO: 8. The mAB of the construct may comprise SEQ ID NO: 9.

The construct is such that its intrinsic mAB, or fragment thereof, may bind its target when the CM of the construct is cloven; as may be measured using a FACS analysis.

The current invention also provides a polynucleotide that encodes the construct of the invention and an isolated cell that is capable of expressing the construct of the invention.

Furthermore, the current invention provides a method of treating, in a subject in need thereof, the co-morbidities associated with the disruption of a blood vessel, namely bleeding and inflammation, by means of the use of the construct of the current invention.

### BRIEF DESCRIPTION OF THE DRAWINGS

FIG. 1: The nucleotide- and amino acid sequences representing A) the EP-FpA-PB1.3 HC-V, B) PB1.3 HC-V and C) PB1.3 LC-V are shown. The italic sequences are signal peptide sequences, the bold sequence represents the PB1.3 mAb binding epitope (EP), the underlined sequences represent the thrombin-cleavable linker sequence. Underlined and italic sequences represent restriction enzyme recognition sites.

FIG. 2: Plasmid Maps of pTT-EP-FpA-PB1.3 HC (2A), pTT-PB1.3 HC (2B) and pTT-PB1.3 LC (2C) are shown. Abbreviations are the following: AmpR: Ampicillin resistance gene; pMB1 ori: origin of replication in E. coli; pCMV: cytomegalovirus promoter; TPL: Tripartite leader; enh MLP: adeno major late promoter enhancer; oriP: origin of replication of Epstein-Barr virus; PolyA: polyadenlyation site; SP: Signal peptide; EP: PB1.3 mAb binding epitope; FpA: thrombin-cleavable linker.

FIG. 3: Results of FACS binding experiments of PB1.3 (3A) and EP-FpA-PB1.3 (3B) to activated and non-activated platelets are shown. The isotype matched control antibody _{CTRL}APC was included in both experiments.

### DESCRIPTION OF THE SEQUENCES

SEQ ID NO: 1 provides the polypeptide sequence of human CD62P (including the signal peptide).

SEQ ID NOs: 2-3 provide the polynucleotide and polypeptide sequences for the EP-FpA-PB1.3 HC-V construct.

SEQ ID NO: 4-5 provide the polynucleotide and polypeptide sequences for the variable domain of the heavy chain of the humanised, anti-CD62P monoclonal antibody, PB1.3.

SEQ ID NOs: 6-7 provide the polynucleotide and polypeptide sequences for the variable domain of the light chain of the humanised, anti-CD62P monoclonal antibody, PB1.3.

SEQ ID NO: 8 provides the polypeptide sequence of the heavy chain of a monoclonal antibody that is capable of binding CD62P.

SEQ ID NO: 9 provides the polypeptide sequence of the light chain of a monoclonal antibody that is capable of binding CD62P.

SEQ ID NOs: 10-11 provide the polynucleotide and polypeptide sequences for tissue factor.

SEQ ID NOs: 12-13 provide the polynucleotide and polypeptide sequences for the extracellular domain of TF.

### DETAILED DESCRIPTION OF THE INVENTION

### CD62P

CD62P (P-Selectin) is a well-known and well-characterized receptor molecule that has been identified on activated platelets and endothelial cells. CD62P is present in the a-granules of resting platelets and in the Weibel-Palade bodies of endothelial cells and can be rapidly translocated to the cell surface upon activation. Upon activation of a platelet, its a-granules fuse with the plasma membrane, translocating the CD62P membrane protein to the cell surface. Surface expression of P-selectin increases 40-50 fold within minutes after platelet activation.

CD62P belongs to the family of selectin adhesion molecules and is expressed by platelets and endothelial cells on stimulation.

It has been demonstrated that CD62P is one of possibly several molecules which link cardiovascular disorders, inflammation and tumour metastasis. For example, it is known that CD62P binding activates leukocytes and endothelial cells via ligands such as P-Selectin glycoprotein ligand-1 (PSGL-1); that platelet-mediated leukocyte rolling depends on CD62P and that endothelial CD62P initiates leukocyte extravasation in immune-mediated diseases such as inflammatory bowel diseases, multiple sclerosis, psoriasis or rheumatoid arthritis.

In terms of the current invention, CD62P may be from any vertebrate, such as any mammal, such as a mouse, a rat, a rabbit, a guinea pig, a pig, a cow, an ape or a human. CD62P may be translated from any naturally occurring genotype or allele that gives rise to a functional protein. A non-limiting example of one human CD62P is the polypeptide sequence of SEQ ID NO: 1.

### Tissue Factor

Tissue Factor is a 263 amino acid, integral membrane glycoprotein receptor. It consists of an extracellular part folded into two compact fibronectin type III-like domains (1-219) that are each stabilized by a single disulfide bond, a transmembrane segment (220-242), and a short cytoplasmic tail (243-263). It forms a tight Ca²⁺-dependent complex with Factor VII/FVIIa.

In terms of the current invention, “tissue factor, or any biologically functional variant or fragment thereof”, may be any polypeptide that is able to bind Factor VII/VII(a), such that blood coagulation is initiated. “Tissue factor” may be derived from any vertebrate animal, such as any mammal, such as a mouse, rat, rabbit, guinea pig, dog, pig, cow, ape or human. “Tissue factor, or any biologically functional variant or fragment thereof” may be the extracellular domain of human tissue factor. “Tissue factor, or any biologically functional variant or fragment thereof” may be any polypeptide that is at least 90%, such as at least 91%, such as at least 92%, such as at least 93%, such as at least 94%, such as at least 95%, such as at least 96%, such as at least 97%, such as at least 98%, such as at least 99% identical to the polypeptide sequence of tissue factor according to SEQ ID NO: 11 or SEQ ID NO: 13. “Tissue factor, or any biologically functional variant or fragment thereof” may be any polypeptide that is at least 90%, such as at least 91%, such as at least 92%, such as at least 93%, such as at least 94%, such as at least 95%, such as at least 96%, such as at least 97%, such as at least 98%, such as at least 99% identical to the polypeptide sequence of the extracellular domain of tissue factor. “Tissue factor, or any biologically functional variant or fragment thereof” may be any polypeptide that is able to function as co-factor for FVII and FVIIa. Hence, “tissue factor or any biologically functional variant or fragment thereof” may be any polypeptide that is able to stimulate the amidolytic activity of FVIIa. Said “tissue factor, or any biologically functional variant or fragment thereof” may be the extracellular domain of Tissue Factor AA1-219 (SEQ ID NO: 13). “Tissue Factor polypeptide” may be a polypeptide comprising the soluble extracellular domain of Tissue Factor, i.e. amino acids 1-219 (in the following referred to as sTF or sTF(1-219)), or a functional variant or truncated form thereof. Preferably, the Tissue Factor polypeptide at least comprises a fragment corresponding to the amino acid sequence 6-209 of Tissue Factor. Examples hereof are sTF(6-209), sTF(1-209) and sTF(1-219).

In accordance with the current invention, “tissue factor, or any biologically functional variant or fragment thereof” may have any one or more of the features listed above.

### Identity

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following: Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

### Construct

The current invention provides a construct that comprises a ligand, such as a monoclonal antibody (mAB) or fragment thereof, a cleavable motif (CM) and a polypeptide that includes the epitope motif (EM) for said mAB or fragment thereof. Said ligand component of the construct may be capable of binding a selectin, such as CD62P. The construct of the current invention is preferably engineered such that it is inert when its constituent CM is uncloven and such that its constituent mAB, or fragment thereof, is capable of binding its target when the CM is cloven.

The term “construct”, as used herein, refers to a polypeptide or a polynucleotide molecule that is characterized by covalently and operably linked elements. For example, a construct can refer to a construct that comprises at least a mAB, a CM and an EM which are operably linked to provide the activatable construct described herein, as well as nucleic acids encoding such constructs. The construct of the invention may also comprise other elements, such as tissue factor or a fragment thereof.

### Ligand

The term “ligand” refers to any substance that is able to bind to and form a complex with a biomolecule, in order to serve a biological purpose. In one sense of the term, it is a signal triggering molecule binding to a site on a target protein by means of intermolecular forces such as ionic bonds, hydrogen bonds and Van der Waals forces. The association of a ligand with said biomolecule is usually reversible. Binding of a naturally occurring ligand to its counterpart receptor alters the chemical conformation of the receptor protein.

The ligand of the current invention may be any naturally occurring or synthetic ligand that binds a selectin on an activated endothelial cell, platelet or leukocyte, or the ligand may be an antibody, or fragment thereof, that has been raised against a selectin. The ligand of the current invention may or may not result in a change in the chemical conformation of said selectin. Furthermore, the ligand of the current invention may or may not result in intracellular signaling, as a result of binding to its target selectin. Hence, the ligand of the current invention utilises a naturally occurring receptor in order to achieve the effect that is unique to and provided by the current invention.

The ligand of the current invention may not bind a selectin to the effect that the biological effect of its natural ligand is mimicked. An analogue of CD62P's ligand, PSGL-1, may thus be a ligand according to the current invention, provided that it blocks activation of CD62P rather than causing activation of the same.

Alternatively, the ligand of the current invention may be an monoclonal antibody, or a fragment thereof, that is capable of binding a selectin, such as CD62P.

### Antibodies

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

An antibody of the invention may be a monoclonal antibody or a polyclonal antibody. In one embodiment, an antibody of the invention is a monoclonal antibody. An antibody of the invention may be a chimeric antibody, a CDR-grafted antibody, a human or humanised antibody or an antigen binding portion of any thereof. For the production of both monoclonal and polyclonal antibodies, the experimental animal is suitably a mammal such as a goat, rabbit, rat or mouse.

Polyclonal antibodies are antibodies that are derived from different B cell lines. A polyclonal antibody may comprise a mixture of different immunoglobulin molecules that are directed against a specific antigen. The polyclonal antibody may comprise a mixture of different immunoglobulin molecules that bind to one or more different epitopes within an antigen molecule. Polyclonal antibodies may be produced by routine methods, such as immunisation of a suitable animal with the antigen of interest. Blood may be subsequently removed from the animal and the immunoglobulin fraction purified.

Monoclonal antibodies are immunoglobulin molecules that are identical to each other and have a single binding specificity and affinity for a particular epitope. Monoclonal antibodies (mAbs) of the present invention can be produced by a variety of techniques, including conventional monoclonal antibody methodology e.g., the standard somatic cell hybridization technique of Kohler and Milstein (1975) Nature 256: 495, or viral or oncogenic transformation of B lymphocytes. The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

To generate hybridomas producing monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. The antibody secreting hybridomas can be replated, screened again, and if still positive for suitable IgG, the monoclonal antibodies can be subcloned at least twice by limiting dilution. The stable subclones can then be cultured in vitro to generate small amounts of antibody in tissue culture medium for characterization.

The term “antigen-binding portion” of an antibody refers to one or more fragments of an antibody that retain the ability to specifically bind to an antigen, such as CD62P or another target protein as described herein. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody of the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

Antibodies of the invention can be tested for binding to the target protein by, for example, standard ELISA or Western blotting. An ELISA assay can also be used to screen for hybridomas that show positive reactivity with the target protein. The binding specificity of an antibody may also be determined by monitoring binding of the antibody to cells expressing the target protein, for example, by flow cytometry.

The specificity of an antibody of the invention for the target protein may be further studied by determining whether or not the antibody binds to other proteins. For example, where it is desired to produce an antibody that specifically binds CD62P or a particular part, e.g. epitope, of CD62P, the specificity of the antibody may be assessed by determining whether or not the antibody also binds to other molecules or modified forms of CD62P that lack the part of interest.

Polypeptide or antibody “fragments” according to the invention may be made by truncation, e.g. by removal of one or more amino acids from the N and/or C-terminal ends of a polypeptide. Up to 10, up to 20, up to 30, up to 40 or more amino acids may be removed from the N and/or C terminal in this way. Fragments may also be generated by one or more internal deletions.

An antibody that forms part of the construct of the current invention may have the ability to bind a selectin on the surface of an activated endothelial cell, platelet or leukocyte. Said antibody may be able to bind a selectin such that the ligand which would normally bind said selectin is prevented from binding it. Thus, said mAB or fragment thereof may block the activity of a ligand that normally binds a selectin. An antibody of the invention may be, or may comprise, a fragment of an anti-CD62P antibody or a variant thereof. A mAB that forms a part of the construct according to the current invention may be that described in U.S. Pat. No. 5,800,815, which is herein incorporated by reference in its entirety. Similarly, the construct of the current invention may comprise a fragment of the mAB described in U.S. Pat. No. 5,800,815. A ligand that may be blocked by the construct of the current invention may be PSGL-1. The antibody of the invention may be or may comprise an antigen binding portion of this antibody, or a variant thereof, as discussed further above. For example, the antibody of the invention may be a Fab fragment of this antibody, or a variant thereof, or may be a single chain antibody derived from this antibody or a variant thereof.

### Epitope

The term “epitope”, as used herein, is defined in the context of a molecular interaction between an “antigen binding polypeptide” (Ab) and its corresponding “antigen” (Ag). As used herein, the term Ab comprises an antibody or a fragment thereof, such as, but not limited to, a Fab, F(ab′)₂ or a Fv fragment, that specifically binds the corresponding Ag. The term Ag refers to the molecular entity used for immunization of an immunocompetent vertebrate to produce the Ab that recognizes the Ag. Herein, Ag is termed more broadly and is generally intended to include molecules that are specifically recognized by the Ab, thus including fragments or mimics of the molecule used in the immunization process for raising the Ab.

Generally, the term “epitope” refers to the area or region on an Ag to which an Ab specifically binds.

A protein epitope may comprise amino acid residues in the Ag directly involved in binding to a Ab (also called the immunodominant component of the epitope) and other amino acid residues, which are not directly involved in the binding, such as amino acid residues on the Ag which are effectively blocked by the Ab (in other words, the amino acid residue is within the “solvent-excluded surface” and/or “footprint” of the Ab). The term epitope herein includes both types of binding sites on any particular region of a selectin, such as CD62P, that specifically binds to an anti-selectin antibody, or another selectin-specific construct component according to the invention, unless otherwise stated (e.g., in some contexts the invention relates to antibodies that bind directly to particular amino acid residues). Any selectin may comprise a number of different epitopes, which may include, without limitation, (1) linear peptide antigenic determinants, (2) conformational antigenic determinants which consist of one or more non-contiguous amino acids located near each other in the mature receptor conformation; and (3) post-translational antigenic determinants which consist, either in whole or part, of molecular structures covalently attached to a given selectin (such as carbohydrate groups).

The epitope for a given Ab/Ag pair can be defined and characterized at different levels of detail using a variety of experimental and computational epitope mapping methods. The experimental methods include mutagenesis, X-ray crystallography, Nuclear Magnetic Resonance (NMR) spectroscopy, Hydrogen deuterium eXchange Mass Spectrometry (HX-MS) and various competition binding methods. As each method relies on a unique principle the description of an epitope is intimately linked to the method by which it has been determined. Thus, the epitope for a given Ab/Ag pair will be defined differently depending on the epitope mapping method employed.

At its most detailed level, an epitope can be defined by the atomic contacts that are important for the interaction between the Ag and the Ab. At a less detailed level the epitope can be characterized by the amino acid residues that it comprises. At a further less detailed level the epitope can be characterized through function, e.g. by competition binding with other Abs.

In the context of an X-ray derived crystal structure defined by atomic coordinates of a complex between an Ab, e.g. an Ab Fab fragment, and its Ag, the term epitope is herein, unless otherwise specified or contradicted by context, specifically defined as residues characterized by having a heavy atom (i.e. a non-hydrogen atom) within a distance of 4 Å from a heavy atom in the Ab.

From the fact that descriptions and definitions of epitopes, dependant on the epitope mapping method used, are obtained at different levels of detail, it follows that comparison of epitopes for different Ab on the same Ag can similarly be conducted at different levels of detail.

Epitopes described on the amino acid level, e.g. determined from an X-ray structure, are said to be identical if they contain the same set of amino acid residues. Epitopes are said to overlap if at least one amino acid is shared by the epitopes. Epitopes are said to be separate (unique) if no amino acid residue is shared by the epitopes.

Epitopes characterized by competition binding are said to be overlapping if the binding of the corresponding Ab's are mutually exclusive, i.e. binding of one Ab excludes simultaneous binding of the other Ab. The epitopes are said to be separate (unique) if the Ag is able to accommodate binding of both corresponding Ab's simultaneously.

### Paratope

Generally, the term “paratope” refers to the area or region on the Ab to which an Ag specifically binds.

In the context of an X-ray derived crystal structure defined by atomic coordinates of a complex between an Ab, e.g. an Ab Fab fragment, and its Ag, the term paratope is herein, unless otherwise specified or contradicted by context, specifically defined as Ag residues characterized by having a heavy atom (i.e. a non-hydrogen atom) within a distance of 4 Å from a heavy atom of the epitope.

### Epitope Motif

The epitope motif (EM) of a construct according to the current invention generally refers to an amino acid sequence that is positioned in the construct such that in an uncleaved state, even in the presence of a target for the monoclonal antibody (mAB), or fragment thereof, the EM interferes with binding of said mAB or mAB fragment to its target. However, in the cleaved state of the construct, EM binding to said target is reduced, thereby allowing greater access of said mAB or fragment thereof to the target and the provision of target binding. Thus, the EM is one which, when the construct is uncleaved, prevents the mAB, or fragment thereof, from binding to its target, but does not substantially or significantly interfere or compete with binding to the target of the mAB, once the construct is cleaved. Thus, the combination of the EM and the CM provides for an activatable construct, with the EM decreasing binding of target when the construct is uncleaved, and cleavage of the CM by protease providing for increased binding of target.

The structural properties of the EM will vary according to a variety of factors such as the minimum amino acid sequence required for interference with mAB binding to its target, the paratope/epitope interaction, the length of the CM, whether or not the CM is positioned within the EM, the presence or absence of linkers, the presence or absence of a cysteine within or flanking the mAB, or fragment thereof, that may provide a cysteine-cysteine disulfide bond, and the like.

The C-terminus of said EM may be covalently linked to the N-terminus of said CM. The C-terminus of said EM may be covalently linked to the mAB, or fragment thereof, by means of, for example, cystein chemistry, glycan chemistry, N-terminal chemistry or lysine chemistry. The EM may comprise one or more residues of the target selectin that interact(s) with the CDR region of said mAB, or fragment thereof. The EM may be linear, or it may be a linear assembly of a structural epitope. The EM may prevent said mAB, or fragment thereof, from binding its target, as may be measured by means of binding interaction analyses (such as those described in examples 4 and 5). The EM may comprise an amino acid sequence corresponding to residues 20-39 (SVLQCLATGNWNSVPPECQA) of SEQ ID NO: 3.

The EM can be provided in a variety of different forms. The EM may bind the mAB, or fragment thereof, with the same affinity as its target selectin. The EM may bind the mAB, or fragment thereof, with a lesser affinity than its target selectin, such as to reduce interference of the EM in target-mAB binding when the CM is cloven. The mAB and EM may comprise amino acid sequences which are not naturally-occurring. The mAB and EM may be a binding partner pair of an anti-CD62P mAB, or fragment thereof, and a complete or partial extracellular domain of a selectin such as CD62P, or derivatives thereof, that act as a binding partner for the mAB, or fragment thereof. The mAB or fragment thereof and the EM may also be selected so they are not natural binding partners. For example, the EM may be a modified binding partner for the mAB or fragment thereof. In this case, the EM may contain amino acid modifications that at least slightly decrease its affinity and/or avidity of binding to the mAB such that, following cleavage, the EM does not substantially or significantly interfere with mAB-target binding. Alternatively, the EM may not specifically bind the mAB, or fragment thereof, but rather interferes with mAB-target binding via non-specific interactions such as stearic hindrance.

The EM may comprise fragments of CD62P. Such fragments may comprise up to 200, such as up to 150, such as up to 100, such as up to 75, such as up to 50, such as up to 25, such as 20, such as 19, such as 18, such as 17, such as 16, such as 15, such as 14, such as 13, such as 12, such as 11, such as 10, such as 9, such as 8, such as 7, such as 6, such as 5, such as 4, such as 3, such as 2, such as 1 amino acid(s) of CD62P. These fragments may be a single continuous segment of amino acids. These fragments may also be assembled as a multitude of short continuous segments of amino acids, bound together in a specific order. The fragments may also be combined with any other continuous segment of amino acid(s), which do not belong to the sequence of human CD62P. In one embodiment, such a fragment may be CD62P20-39.

EM candidates may be designed according to structural information obtained by experimental and computational epitope mapping methods. These experimental methods include mutagenesis, X-ray crystallography (XTaI), Nuclear Magnetic Resonance (NMR) spectroscopy, Hydrogen deuterium eXchange Mass Spectrometry (HXMS) and various competition binding methods e.g. Enzyme-Linked Immunosorbent Assay (ELISA), Biacore or Fluorescence Activated Cell Sorting FACS methods. All these methods are well established.

EM candidates may also be identified using evolution-based screening methods such as phage and bacterial display. These methods utilize display of foreign (poly)peptides on the surface of a phage particle or bacterial surface via splicing a gene encoding such a peptide into a gene encoding a phage capsid structural protein or a bacterial protein targeted to the outer membrane or proteins assembled into flagella and fimbrial structures. Using recombinant DNA technology, collections of billions of peptides, protein variants, gene fragment- or cDNA-encoded proteins presented on phage (so-called display libraries) can be constructed. One well-known example is the FliTrx systems in which peptides are presented as constrained insertions within the active site loop of E. coli thioredoxin, which is in turn inserted into a surface-exposed region of the abundant, repeating flagellar protein FIiC. Determination of an antibody's binding specificity, and specific epitope, can then be determined by combining the display method in question with selection and screening procedures using for example Magnetic Activated Cell Sorting (MACS) or Fluorescence Activated Cell Sorting (FACS) methods. In this way (poly)peptides may be selected as epitope motif candidates for the activable construct based on binding specificity for the antibody or fragment hereof in question [Ref: Daugherty, P. S. (2007) Protein engineering with bacterial. Curr Opin Struct Biol 17: 474-80. Bratkovi\check{c}, T. (2010) Progress in phage display: evolution of the technique and its applications. Cellular and Molecular Life Sciences 67: 749-67].

An EM candidate selected by one of the mentioned methods may be further optimized by changing and/or interchanging residues of the (poly)peptide with one or more natural or unnatural amino acid. In this way the specificity and/or affinity may be increased or decreased for the antibody or fragment hereof in question. This may be utilized for designing a specific binding mode for the motif to the antibody or fragment hereof in question reflecting an optimal functionality and/or usage, as for example, for a pharmaceutical composition.

### Binding Affinity

The term “binding affinity” is herein used as a measure of the strength of a non-covalent interaction between two molecules, e.g. an antibody, or fragment thereof, and an antigen. The term “binding affinity” is used to describe monovalent interactions (intrinsic activity), and may be distinguished from “binding avidity” (functional affinity), that refers to the strength of a multivalent interaction.

Binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation, e.g. by the SPR method (Biacore). The rate constants corresponding to the association and the dissociation of a monovalent complex are referred to as the association rate constants kₐ (or kₒₙ) and dissociation rate constant k_{d}. (or k_{off}), respectively. K_{D} is related to kₐ and k_{d} through the equation K_{D}=k_{d}/kₐ.

Following the above definition, binding affinities associated with different molecular interactions, e.g. comparison of the binding affinity of different antibodies for a given antigen, may be compared by comparison of the K_{D} values for the individual antibody/antigen complexes.

Similarly, the specificity of an interaction may be assessed by determination and comparison of the K_{D} value for the interaction of interest, e.g. a specific interaction between an antibody and an antigen, with the K_{D} value of an interaction not of interest.

Typically, the K_{D} for the antibody with respect to the target will be 2-fold, preferably 5-fold, more preferably 10-fold less than K_{D} with respect to the other, non-target molecule such as unrelated material or accompanying material in the environment. More preferably, the K_{D} will be 50-fold less, such as 100-fold less, or 200-fold less; even more preferably 500-fold less, such as 1,000-fold less, or 10,000-fold less.

The value of this dissociation constant can be determined directly by well-known methods, and can be computed even for complex mixtures by methods such as those, for example, set forth in Caceci et al. (Byte 9:340-362, 1984). For example, the K_{D} may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics and binding affinity of the antibody also can be assessed by standard assays known in the art, such as Surface Plasmon Resonance (SPR), e.g. by using a Biacore™ system.

A competitive binding assay can be conducted in which the binding of the antibody to the target is compared to the binding of the target by another ligand of that target, such as another antibody. The concentration at which 50% inhibition occurs is known as the Ki. Under ideal conditions, the Ki is equivalent to K_{D}. The Ki value will never be less than the K_{D}, so measurement of Ki can conveniently be substituted to provide an upper limit for K_{D}.

An antibody of the invention may have a K_{D} for its target of 1×10⁻⁷M or less, 1×10⁻⁸M or less, or 1×10⁻⁹M or less, or 1×10⁻¹⁰M or less, 1×10⁻¹¹M or less, or 1×10⁻¹²M or less. An antibody that specifically binds its target may bind its target with a high affinity, that is, exhibiting a low K_{D} as discussed above, and may bind to other, non-target molecules with a lower affinity. For example, the antibody may bind to non-target molecules with a K_{D} of 1×10⁻⁶M or more, more preferably 1×10⁻⁵ M or more, more preferably 1×10⁻⁴ M or more, more preferably 1×10⁻³ M or more, even more preferably 1×10⁻² M or more. An antibody of the invention is preferably capable of binding to its target with an affinity that is at least two-fold, 10-fold, 50-fold, 100-fold 200-fold, 500-fold, 1,000-fold or 10,000-fold or greater than its affinity for binding to another non-target molecule.

### Cleavable Motif

The cleavable motif (CM) of the construct according to the current invention may comprise an amino acid sequence that can serve as a substrate for a protease, usually an extracellular protease. The CM is positioned in the construct such that when the CM is cleaved by a cleaving agent (e.g., a protease substrate of a CM is cleaved by the protease), in the presence of a target, resulting in a cleaved state, the monoclonal antibody or fragment thereof binds its target, whereas in an uncleaved state, in the presence of its target, binding of the monoclonal antibody, or fragment thereof, to its target is inhibited by the epitope motif (EM). It should be noted that the amino acid sequence of the CM may overlap with or be included within the EM, such that all or a portion of the CM facilitates “masking” of the paratope of the monoclonal antibody, or fragment thereof, when the construct is in the uncleaved conformation.

As discussed above, the CM may be selected based on a protease whose location is the same microenvironment as the desired target of the monoclonal antibody, or fragment thereof, of the construct. A variety of different conditions are known in which a target of interest is co-localized with a protease, where the substrate of the protease is known in the art. For example, the target tissue may be an activated platelet.

As such, where the mAB of an construct is selected such that it is capable of binding a target such as CD62P, a suitable CM will be one which comprises a peptide substrate that is cleavable by a protease that is present at the site of a damaged blood vessel, particularly that is present at elevated levels at the site of a damaged blood vessel as compared to within intact vessels.

The CM may be the cleavage site of a protease, such as the activation peptide of a protease, such as the activation peptide of a serine protease. The CM may be up to 200 amino acids in length, such as up to 150 amino acids, such as up to 100 amino acids, such as up to 75 amino acids, such as up to 50 amino acids, such as up to 25 amino acids, such as 2-20 amino acids, such as 20 amino acids, such as 19 amino acids, such as 18 amino acids, such as 17 amino acids, such as 16 amino acids, such as 15 amino acids, such as 14 amino acids, such as 13 amino acids, such as 12 amino acids, such as 11 amino acids, such as 10 amino acids, such as 9 amino acids, such as 8 amino acids, such as 7 amino acids, such as 6 amino acids, such as 5 amino acids, such as 4 amino acids, such as 3 amino acids, such as 2 amino acids in length. P1′ may be an amino acid selected from the group consisting of A, G, I, L, S and T. P1 of said CM may be an amino acid selected from the group consisting of R and K. In a specific embodiment, P1 of the CM is R. In another specific embodiment, P1 of the CM is K. In one embodiment, P1-P1′ of the CM is RI. In another embodiment, P1-P1′ of the CM is RG. In a third embodiment, P1-P1′ of the CM is RT. In yet another embodiment, P1-P1′ of the CM is RS. P2 may be an amino acid selected from the group consisting of A, I, L and P. P3 may be any amino acid selected from the group consisting of A, R, N, D, C, E, Q, G, H, I, L, K, M, F, P, S, T, W, Y and V. P4 may be an amino acid selected from the group consisting of A, Q, G, I, L, F, W and V. The CM may be fibrinopeptide A (FpA), or a fragment thereof, such as the amino acid sequence corresponding to residues 40-49 (DFLAEGGGVR) of SEQ ID NO: 3. The CM may be fibrinopeptide B (FpB), or a fragment thereof. The CM may be the activation peptide of Factor V. The CM may be the activation peptide of Factor VII. The CM may be the activation peptide of FVIII. The CM may be the activation peptide of PAR1. The CM may be the activation peptide of PAR2.

### Cleaving Agent

According to the current invention, a cleaving agent may be any protease that is present and active in the same biological microenvironment as the target of the construct of the current invention and which is capable of reducing the cleavable motif. Thus, said protease may be present in the proximity of an activated platelet or a damaged blood vessel. The CM may be cloven by an enzyme selected from the group consisting of thrombin, Factor VIIa (FVIIa), Factor IXa (FIXa), Factor Xa (FXa), Factor XIa (FXIa), Factor XIIa (FXIIa), kallikrein, activated protein C (APC), plasmin, tissue Plasminogen Activator (tPA) and urokinase-type Plasminogen Activator (uPA). The CM may be cloven by thrombin. The CM may be cloven by FVIIa. The CM may be cloven by FIXa. The CM may be cloven by FXa. The CM may be cloven by FXIa. The CM may be cloven by FXIIa. The CM may be cloven by kallikrein. The CM may be cloven by APC. The CM may be cloven by plasmin. The CM may be cloven by tPA. The CM may be cloven by uPA.

The terms “protease”, “proteinase” and “enzyme capable of cleaving a polypeptide” are used interchangeably herein to refer to any enzyme, e.g., an activated serine protease, which is capable of hydrolysing peptide bonds.

### Production of Nucleic Acid Sequences Encoding Candidate Constructs

Production of candidate constructs for use in the screening methods can be accomplished using methods known in the art. Polypeptide display, single chain antibody display, antibody display and antibody fragment display are methods well know in the art. In general, an element of a construct (such as the EM) to be varied in the candidate construct library is selected for randomization. The candidate constructs in the library can be fully randomized or biased in their randomization, e.g. in nucleotide/residue frequency generally or in position of amino acid(s) within an element. By “randomized” it is meant that any genetically-encodable amino acid can be provided at any given position within a randomized amino acid sequence. An amino acid sequence of an element of a construct that is to be optimized can also be partially randomized. For example, the construct element (e.g., candidate epitope motif) can be partially randomized so as to provide for only a subset of amino acids at a selected position (e.g., to provide for a flexible linker at a selected position in the amino acid sequence, to provide for an amino acid residue of a desired characteristic (e.g., hydrophobic, polar, positively charged, negatively charged, etc.). In another example, the construct element (e.g., candidate epitope motif) can be partially randomized so that one or more residues within the otherwise randomized amino acid sequence is selected and held as invariable among a population or subpopulation of construct library members.

Using such methods, candidate constructs having a variety of different possible combinations of amino acid sequence over the length of the amino acid sequence of an element(s) to be varied can be generated, thus providing a library of randomized candidate constructs. As such, in some embodiments, the library of candidate constructs can be fully randomized, with no sequence preferences or constants at any position of an element(s) to be optimized. In other embodiments, the library of candidate peptides is biased. That is, some positions within the sequence are either held constant, or are selected from a limited number of possibilities. For example, in one embodiment, the nucleotides or amino acid residues are randomized within a defined class, for example, of hydrophobic amino acids, hydrophilic residues, sterically biased (either small or large) residues, towards the creation of cysteines, for cross-linking.

### Formation of the Chimeric Construct

DNA fragments may be ligated in accordance with conventional techniques known in the art. Such techniques include the use of restriction enzymes to digest DNA fragments, DNA polymerases and nucleotides to fill in sticky ends to form blunt ends, alkaline phosphatase to avoid undesired ligations, and ligases to join fragments.

The mAB, or fragment thereof, the CM and the EM of the construct of the invention may be joined together to form a single DNA segment or they may be maintained as separate segments by themselves. The constructs may be introduced into a cell by transformation, in conjunction with a gene allowing for selection, wherein the construct will become integrated into the host genome. Usually, the construct will be part of a vector having a replication system recognized by the host cell.

### Expression Vectors

Expression vehicles for producing the constructs of the invention include vectors such as plasmids. In general, such vectors contain control sequences that allow expression in various types of hosts, including but not limited to prokaryotes, yeasts, fungi, plants and higher eukaryotes. Suitable expression vectors containing the desired coding and control sequences may be constructed using recombinant DNA techniques known in the art, many of which are described in Sambrook, et al., Molecular Cloning: A Laboratory Manual, Second Edition, Cold Spring Harbor Laboratory, Cold Spring Habor, N.Y. (1989).

An expression vector as contemplated by the present invention may be the pTT vector described in Durocher, Y. et al., (2002) Nucleic Acid Res, 30: E9. Such an expression vector is at least capable of directing the replication, and preferably the expression, of the nucleic acids of the present invention. One class of vectors utilizes DNA elements that provide autonomously replicating extrachromosomal plasmids derived from animal viruses (such as the bovine papilloma virus, polyomavirus, adenovirus, or SV40 virus). A second class of vectors relies upon the integration of the desired gene sequences into the host cell chromosome.

Expression vectors useful in the present invention include sequences that control the replication and expression of the subject DNA sequence. Typically, the expression vector contains an origin of replication, a promoter located 5′ to (i.e., upstream of) the DNA sequence to be expressed, and a transcription termination sequence. Suitable origins of replication include, for example, the Col E1, the SV40 viral and the M13 origins of replication. Suitable termination sequences include, for example, the bovine growth hormone, SV40, lac Z and the Autographa californica multiple nuclear polyhedrosis virus (AcMNPV) polyhedral polyadenylation signals. Suitable promoters include, for example, the immunoglobulin H chain promoter, the cytomegalovirus promoter, the lac Z promoter, the gal 10 promoter and the AcMNPV polyhedral promoter.

The expression vectors may also include other regulatory sequences for optimal expression of the desired product. Such sequences include stability leader sequences, which provide for stability of the expression product; secretory leader sequences, which provide for secretion of the expression product; enhancers, which upregulate the expression of the DNA sequence; and restriction enzyme recognition sequences, which provide sites for cleavage by restriction endonucleases. All of these materials are known in the art and are commercially available. See, for example, Okayama, Mol. Cell. Biol., 3 280 (1983).

A suitable expression vector may also include marking sequences, which allow phenotypic selection of transformed host cells. Such a marker may provide prototrophy to an auxotrophic host, biocide resistance (e.g., antibiotic resistance) and the like. The selectable marker gene can either be directly linked to the DNA gene sequences to be expressed, or introduced into the same cell by co-transfection. Examples of selectable markers include neomycin, ampicillin and hygromycin resistance.

### Host Cells

The present invention also concerns host cells containing one or more expression vectors, such as a plasmid, that comprise a DNA sequence encoding the construct of the current invention.

When using co-transfection with a light chain expression vector, many different prokaryotic and eukaryotic host cells may be employed. Suitable prokaryotic host cells include, for example, E. coli strains HB101, DH5α and XL1 Blue. Suitable eukaryotic host cells include, for example, insect cells such as Spodoptera frugiperda and fungal cells such as Pichia pastoris and Saccharomyces cerevisiae cells.

Other suitable eukaryotic cells are mammalian cells, grown in vitro in tissue culture or in animals. Mammalian cells may provide post-translational modification to immunoglobulin protein molecules, including correct folding or glycosylation at correct sites. Preferred mammalian cell lines include the CHO (DUKX cells (Urlaub and Chasin, Proc. Natl. Acad. Sci. USA 77:4216-4220, 1980), H1 and ATCC CCL 61 cell lines), COS-1 (ATCC CRL 1650), baby hamster kidney (BHK) and HEK293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982), hereinafter referred to as BHK 570 cells. The BHK 570 cell line is available from the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used, including Rat Hep I (Rat hepatoma; ATCC CRL 1600), Rat Hep II (Rat hepatoma; ATCC CRL 1548), TCMK (ATCC CCL 139), Human lung (ATCC HB 8065) and NCTC 1469 (ATCC CCL 9.1) cells.

A cell that may express a construct of the current invention may be separated by FACS, immunochromatography or, where the detectable label is magnetic, by magnetic separation. As a result of the separation, a cell population is enriched for those that exhibit the desired characteristic, for example, constructs that exhibit binding to a selectin, such as CD62P, following their cleavage, or that have decreased or no detectable binding to target in the absence of cleavage.

Selection of candidate constructs having bound a detectably labeled target can be accomplished using a variety of techniques known in the art. For example, flow cytometry (e.g., FACS(R)) methods can be used to sort detectably labeled candidate constructs from unlabeled candidate constructs. Flow cytometry methods can be implemented to provide for more or less stringent requirements in separation of the population of candidate constructs, for example, by modification of gating to allow for “dimmer” or to require “brighter” cell populations in order to be separated into the second population for further screening.

### Methods of Screening for Constructs of the Invention

The present disclosure provides methods of identifying the activatable constructs of the current invention. Generally, methods include contacting a plurality of candidate constructs with a target that is capable of binding the monoclonal antibody, or fragment thereof, of the cloven candidate construct, and a protease that is capable of cleaving the cleavable motif of said construct; selecting a first population of members which bind to the target when exposed to protease, contacting said first population with the target in the absence of the protease, and selecting a second population of members from said first population by depleting from said first population members that bind the target in the absence of the protease, wherein said method provides for selection of candidate constructs which exhibit decreased binding to the target in the absence of the protease as compared to target binding in the presence of the protease.

In general, the method for screening for candidate constructs having the desired activatable phenotype is accomplished through a positive screening step (to identify members that bind target following exposure to protease) and a negative screening step (to identify members that do not bind target when not exposed to protease). The negative screening step can be accomplished by, for example, depleting from the population those members that bind the target in the absence of the protease. Library screening methods may be initiated by conducting the negative screening first to select for candidates that do not bind labelled target in the absence of a protease (that is, that do not bind labelled target when intact), and then conducting the positive screening (i.e., treating with protease and selecting for those members which bind labelled target in the cloven state).

### Indications

A construct of the current invention may be used to decrease inflammation at the site of an injury. Furthermore, a construct of the current invention may be engineered such that it is suitable for the treatment of a coagulopathy. For this purpose, the construct of the current invention may comprise tissue factor (SEQ ID NO: 11) or a functional fragment thereof (SEQ ID NO: 13). The term “coagulopathy”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and may be identified by a clinician.

Non-limiting examples of congenital hypocoagulopathies are haemophilia A, haemophilia B, Factor VII deficiency, Factor XI deficiency, von Willebrand's disease and thrombocytopenias such as Glanzmann's thombasthenia and Bernard-Soulier syndrome.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilutional thrombocytopaenia and dilution of clotting factors), hypothermia, consumption of clotting factors and metabolic derangements (acidosis). Fluid therapy and increased fibrinolysis may exaserbate this situation. Said haemorrhage may be from any part of the body.

Haemophilia A with “inhibitors” (that is, allo-antibodies against factor VIII) and haemophilia B with “inhibitors” (that is, allo-antibodies against factor IX) are non-limiting examples of coagulopathies that are partly congenital and partly acquired.

A non-limiting example of an iatrogenic coagulopathy is an overdosage of anticoagulant medication—such as heparin, aspirin, warfarin and other platelet aggregation inhibitors—that may be prescribed to treat thromboembolic disease. A second, non-limiting example of iatrogenic coagulopathy is that which is induced by excessive and/or inappropriate fluid therapy, such as that which may be induced by a blood transfusion.

In one embodiment of the current invention, haemorrhage is associated with haemophilia A or B. In another embodiment, haemorrhage is associated with haemophilia A or B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intraaurally or intraocularly. In another embodiment, haemorrhage is associated with anticoagulant therapy.

Use of said monoclonal antibody of the invention may significantly reduce blood loss.

Use of said monoclonal antibody of the invention may significantly reduce bleeding time.

### Treatment

The term “treatment”, as used herein, refers to the medical therapy of any human or other animal subject in need thereof. Said subject is expected to have undergone physical examination by a medical practitioner or a veterinary medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

Thus, a construct of the invention may be administered parenterally. A construct of the invention may be administered intravenously. A construct of the invention may be administered intramuscularly. A construct of the invention may be administered subcutaneously. A construct of the invention may be administered prophylactically. A construct of the invention may be administered therapeutically (on demand).

### EMBODIMENTS

The following is a non-limiting list of embodiments of the present invention:

1. A construct comprising a (i) a monoclonal antibody, or a fragment thereof, (ii) a cleavable motif and (iii) a polypeptide sequence that comprises the epitope motif of (i) or a variant thereof.

2. The construct according to embodiment 1, further comprising tissue factor or a biologically functional variant or fragment thereof.

3. The construct according to any one of embodiments 1-2, wherein said integral monoclonal antibody, or fragment thereof, is capable of binding a receptor on a cell selected from the group consisting of an activated platelet, an activated endothelial cell and a leukocyte.

4. The construct according to any one of embodiments 1-3, wherein said integral monoclonal antibody, or fragment thereof, is capable of binding a receptor on an activated platelet.

5. The construct according to any one of embodiments 1-4, wherein said integral monoclonal antibody, or fragment thereof, is not capable of binding a receptor on a resting platelet.

6. The construct according to any one of embodiments 1-5, wherein said integral monoclonal antibody, or fragment thereof, is capable of binding a receptor on an activated endothelial cell.

7. The construct according to any one of embodiments 3-6, wherein said receptor is a selectin.

8. The construct according to any one of embodiments 3 or 7, wherein said receptor is an E-, a P- or an L-selectin.

9. The construct according to embodiment 8, wherein said selectin is CD62P.

10. The construct according to any one of embodiments 1-9, wherein the C-terminus of said cleavable motif is covalently linked to the N-terminus of the heavy chain of said monoclonal antibody, or fragment thereof.

11. The construct according to any one of embodiments 1-10, wherein the C-terminus of said cleavable motif is covalently linked to the N-terminus of the light chain of said monoclonal antibody or fragment thereof.

12. The construct according to any one of embodiments 1-11, wherein P1 of said cleavable motif may be an amino acid selected from the group consisting of R and K.

13. The construct according to any one of embodiments 1-12, wherein said cleavable motif is the cleavage site of a protease.

14. The construct according to embodiment 13, wherein said cleavable motif is the activation peptide of a protease.

15. The construct according to embodiment 14, wherein said protease is a serine protease.

16. The construct according to any one of embodiments 12-15, wherein said cleavable motif may be cloven by an enzyme selected from the group consisting of thrombin, Factor VIIa (FVIIa), Factor IXa (FIXa), Factor Xa (FXa), Factor XIa (FXIa), Factor XIIa (FXIIa), kallikrein, activated protein C (APC), plasmin, tissue Plasminogen Activator (tPA) and urokinase-type Plasminogen Activator (uPA).

17. The construct according to any one of embodiments 12-16, wherein said cleavable motif is approximately 2-20 amino acids in length, such as 20 amino acids, such as 19 amino acids, such as 18 amino acids, such as 17 amino acids, such as 16 amino acids, such as 15 amino acids, such as 14 amino acids, such as 13 amino acids, such as 12 amino acids, such as 11 amino acids, such as 10 amino acids, such as 9 amino acids, such as 8 amino acids, such as 7 amino acids, such as 6 amino acids, such as 5 amino acids, such as 4 amino acids, such as 3 amino acids, such as 2 amino acids in length.

18. The construct according to any one of embodiments 15-17, wherein P2 may be an amino acid selected from the group consisting of A, I, L and P.

19. The construct according to any one of embodiments 15-18, wherein P2 may be any amino acid selected from the group consisting of A, R, N, D, C, E, Q, G, H, I, L, K, M, F, P, S, T, W, Y and V.

20. The construct according to any one of embodiments 15-19, wherein P4 may be an amino acid selected from the group consisting of A, Q, G, I, L, F, W and V.

21. The construct according to any one of embodiments 15-20, wherein P1′ may be an amino acid selected from the group consisting of A, G, I, L, S and T.

22. The construct according to embodiment 14, wherein said cleavable motif is fibrinopeptide A (FpA), or a fragment thereof, such as the amino acid sequence corresponding to residues 40-49 (DFLAEGGGVR) of SEQ ID NO: 3.

23. The construct according to embodiment 22, wherein P1 of said cleavable motif is R.

24. The construct according to any one of embodiments 22-23, wherein P1-P1′ of said cleavable motif is RI.

25. The construct according to any one of embodiments 22-24, wherein P1-P1′ of said cleavable motif is RG.

26. The construct according to embodiment 14, wherein said cleavable motif is fibrinopeptide B (FpB), or a fragment thereof.

27. The construct according to embodiment 26, wherein P1 of said cleavable motif is R.

28. The construct according to any one of embodiments 26-27, wherein P1-P1′ of said cleavable motif is RG.

29. The construct according to embodiment 14, wherein said cleavable motif is the activation peptide of Factor V.

30. The construct according to embodiment 29, wherein P1 of said cleavable motif is R.

31. The construct according to any one of embodiments 29-30, wherein P1-P1′ of said cleavable motif is RT.

32. The construct according to any one of embodiments 29-31, wherein P1-P1′ of said cleavable motif is RS.

33. The construct according to embodiment 15, wherein said cleavable motif is the activation peptide of Factor VII.

34. The construct according to embodiment 33, wherein P1 of said cleavable motif is R.

35. The construct according to any one of embodiments 33-34, wherein P1 of said cleavable motif is K.

36. The construct according to embodiment 14, wherein said cleavable motif is the activation peptide of FVIII.

37. The construct according to embodiment 36, wherein P1 of said cleavable motif is R.

38. The construct according to embodiment 14, wherein said cleavable motif is the activation peptide of PAR1.

39. The construct according to embodiment 38, wherein P1 of said cleavable motif is R.

40. The construct according to any one of embodiments 38-39, wherein P1-P1′ of said cleavable motif is RS.

41. The construct according to embodiment 14, wherein said cleavable motif is the activation peptide of PAR2.

42. The construct according to embodiment 41, wherein P1 of said cleavable motif is R.

43. The construct according to any one of embodiments 41-42, wherein P1-P1′ of said cleavable motif is RS.

44. The construct according to any one of embodiments 1-43, wherein the C-terminus of said epitope motif is covalently linked to the N-terminus of said cleavable motif.

45. The construct according to embodiment 44, wherein said epitope motif is also covalently linked to the monoclonal antibody, or fragment thereof.

46. The construct according to any one of embodiments 1-45, wherein said epitope motif comprises one or more residues of the target receptor that interact(s) with the CDR region of said monoclonal antibody, or fragment thereof.

47. The construct according to embodiment 46, wherein said epitope motif is linear.

48. The construct according to embodiment 46, wherein said epitope motif is a linear assembly of a structural epitope.

49. The construct according to any one of embodiments 1-48, wherein said epitope motif prevents said monoclonal antibody or fragment thereof from binding its target, as may be measured by means of binding interaction analyses (such as those described in examples 4 and 5).

50. The construct according to embodiment 49, wherein said epitope motif comprises an amino acid sequence corresponding to residues 20-39 (SVLQCLATGNWNSVPPECQA) of SEQ ID NO: 3.

51. The construct according to any one of embodiments 1-50, wherein said monoclonal antibody or fragment thereof may bind its target when said cleavable motif is cloven, as may be measured using a fluorescence-activated cell sorting (FACS) analysis (such as that described in example 4).

52. The construct according to any one of embodiments 1-51, wherein said monoclonal antibody comprises SEQ ID NO: 8.

53. The construct according to any one of embodiments 1-52, wherein said monoclonal antibody is comprises SEQ ID NO: 9.

54. A polynucleotide that encodes the construct according to any one of embodiments 1-53.

55. A vector that comprises the polynucleotide according to embodiment 54.

56. A plasmid that comprises the polynucleotide according to embodiment 54.

57. An isolated cell that comprises the polynucleotide according to embodiment 54.

58. An isolated cell that comprises the vector according to embodiment 55.

59. An isolated cell that comprises the plasmid according to embodiment 56.

60. The isolated cell according to embodiment 59, wherein said cell is eukaryotic.

61. The eukaryotic cell according to embodiment 60, which is mammalian.

62. The mammalian cell according to embodiment 61, which is selected from the group consisting of a CHO cell, a BHK cell and a HEK cell.

63. The cell according to any one of embodiments 57-62 that is capable of expressing the construct according to any one of embodiments 1-53.

64. A pharmaceutical composition comprising a construct according to any one of embodiments 1-53 and a pharmaceutically acceptable carrier.

65. Use of the construct according to any one of embodiments 1-53, for the treatment of the co-morbidities associated with the disruption of a blood vessel, namely bleeding and inflammation, in a subject in need thereof.

66. Use of the construct according to any one of embodiments 1-53 for the treatment of a coagulopathy.

67. Use of the construct according to any one of embodiments 1-53 for inhibiting proliferation of arteriosclerotic foam cells.

68. Use of the construct according to any one of embodiments 1-53 for the treatment of inflammation.

69. Use of the construct according to any one of embodiments 1-53 for the prevention of metastasis of malignantly proliferating cells.

70. A method of treating the co-morbidities associated with the disruption of a blood vessel, notably bleeding and inflammation, by means of administering to a subject in need thereof a construct according to any one of embodiments 1-53.

71. A method of treating a coagulopathy, comprising administering to a subject in need thereof the construct according to any one of the embodiments 1-53.

72. A method of treating inflammation, comprising administering to a subject in need thereof the construct according to any one of the embodiments 1-53.

73. A method of treating cancer, comprising administering to a subject in need thereof the construct according to any one of the embodiments 1-53.

The invention is further illustrated by the following examples which are not to be construed as limiting the scope of protection. The features disclosed in the foregoing description and in the following examples may, both separately and in any combination thereof, be material for realising the invention in its diverse forms.

### EXAMPLES

### Example 1

### Development of Expression Constructs Encoding EP-FpA-antiCD62P HC, antiCD62P HC and antiCD62 LC

The LC and HC variable domain amino acid sequences for the antiCD62P mAb (designated PB1.3 HC-V and PB1.3LC-V) together with the PB1.3 binding epitope amino acid sequence (designated EP) were obtained from U.S. Pat. No. 5,800,815. The amino acid sequence of the thrombin-cleavable linker (designated FpA) was based on the amino acid sequence at the thrombin cleavage site in human fibrinogen alpha chain (retrieved from public available databases).

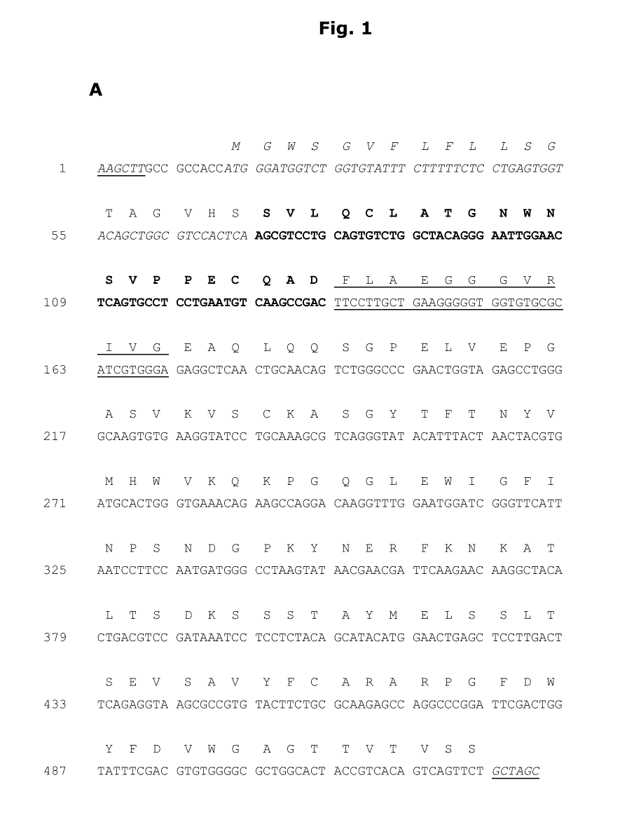

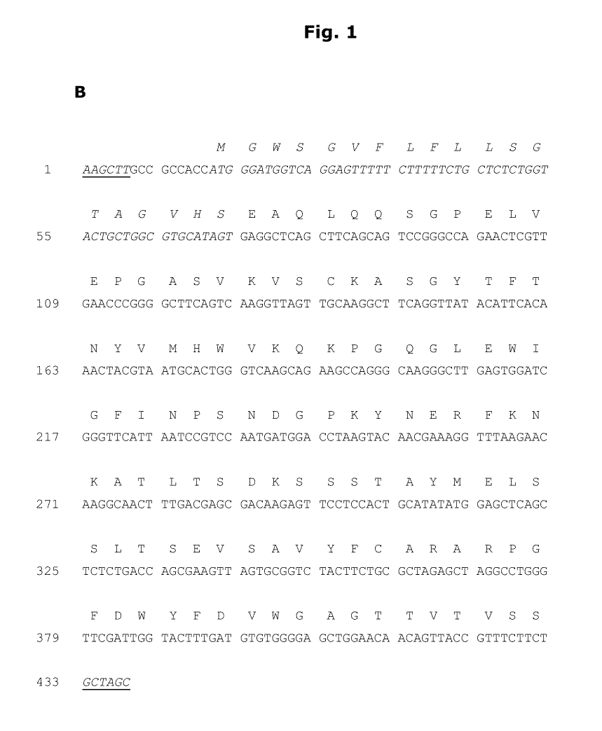

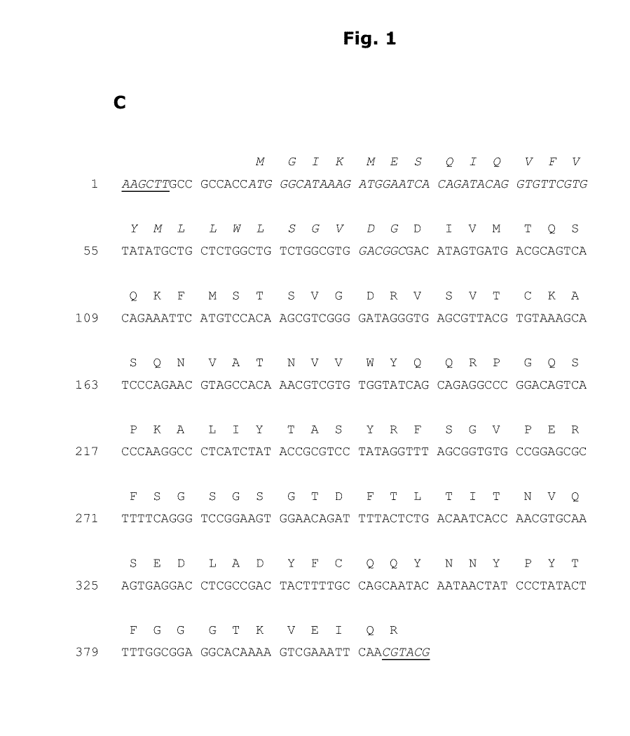

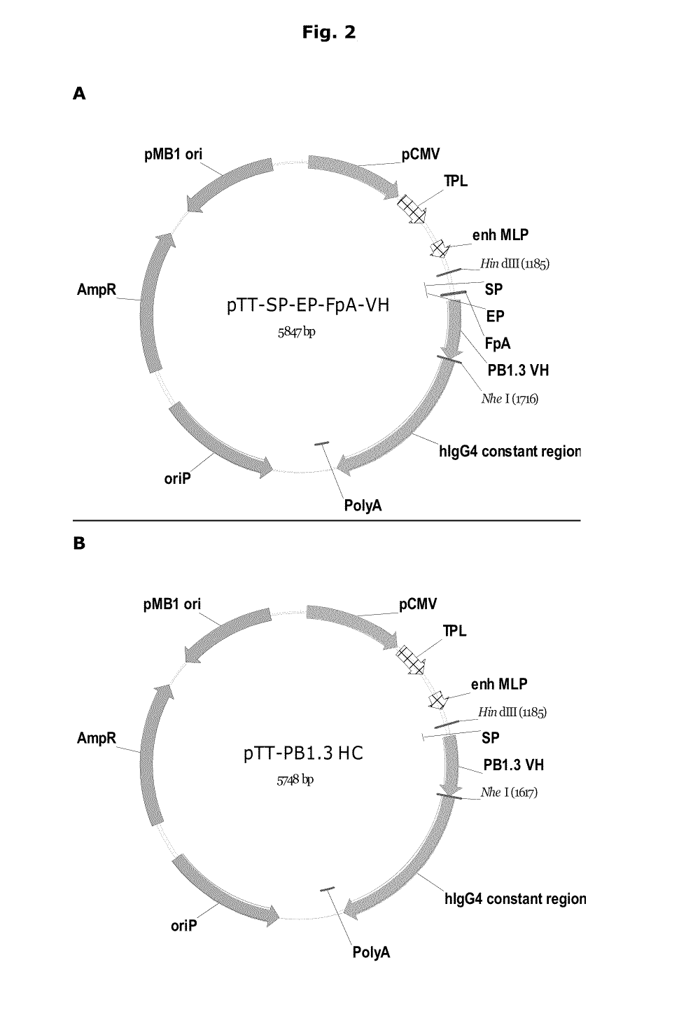

DNA sequences encoding 1) the EP-FpA-PB1.3 HC-V, 2) PB1.3 HC-V or encoding 3) PB1.3 LC-V were synthesized at external CRO, Geneart Inc, CA, USA. The synthesized DNA sequences were made containing a 5′ end HindIII site (AAGCTT) and a kozak sequence (GCCGCCACC) immediately 5′ end to the start methionine and an in-frame 3′ end NheI site (GCTAGC) for the HC-V containing sequences and an in-frame 3′ end BsiWI site (CGTACG) for PB1.3 LC-V sequence (FIGS. 1A, 1B and 1C). The PB1.3 HC-V containing DNA sequences (1 and 2) were digested with HindIII and NheI and inserted into the HindIII and NheI sites in a pTT-based expression vector containing the human IgG4 CH1-hinge-CH2-CH3 sequences. The resulting vectors were designated pTT-EP-FpA-PB1.3 HC and pTT-PB1.3 HC, respectively (FIGS. 2A and 2B). The PB1.3 LC-V DNA sequence was digested with HindIII and BsiWI and inserted into the HindIII and BsiWI sites of a pTT-based expression vector containing the human LC kappa constant sequences. The resulting vector was designated pTT-PB1.3 LC (FIG. 2C). The pTT vector is essentially described in Durocher, Y. et al., (2002) Nucleic Acid Res, 30: E9.

Two transient co-transfection experiments were set-up using the following plasmid combinations: 1) pTT-PB1.3 LC+pTT-EP-FpA-PB1.3 HC and 2) pTT-PB1.3 LC+pTT-PB1.3 HC. In each transfection experiment the plasmids were cotransfected in approximately equimolar ratios. HEK293-6E cells were grown in Freestyle HEK293 medium (GIBCO, cat. no. 12338-018) supplemented with 1% P/S (GIBCO cat. no. 15140-122), 0.1% pluronic (GIBCO, cat. no. 24040-032) and 25 ug/mL Geneticin (GIBCO, cat. no. 10131-019) and cells were transfected at a cell density of 1 mill/mL using 293fectin (Invitrogen, cat. no. 12347-019). For each liter of HEK293-6E cells, the transfection was performed by diluting 1 mg of plasmid DNA into 30 mL Optimem (dilution A) and by diluting 1 mL 293fectin into 30 mL Optimem (GIBCO, cat. no. 51985-026, dilution B). Dilution A and B were mixed and incubated at room temperature for 30 minutes. The transfection mix was hereafter added to the HEK293-6E cells and cells were incubated at 37° C. in a humified incubator with orbital rotation (125 rpm). Five days post-tranfection, cells were removed by centrifugation and the two resulting cell culture supernatants containing EP-FpA-PB1.3 mAb and PB1.3 mAb, respectively, were sterile-filtrated prior to purification.

### Example 2

### Purification of EP-FpA-PB1.3 mAb and PB1.3 mAb Constructs

Protein purification of the mAb constructs EP-FpA-PB1.3 mAb and PB1.3 was conducted using the Proten-A-based affinity resin MabSelect SuRe (GE Healthcare, cat. no. 17-5438). The resin was packed in a Tricorn10/100 column to a bed volume of approx. 8 ml. The purifications were conducted using an Äkta Explorer chromatography system (GE Healthcare, cat. no. 18-1112-41). The buffer systems used for the purification step was an equilibration buffer composed of 20 mM NaPhosphate, 150 mM NaCl, pH 7.2 and an elution buffer composed of 10 mM Formic acid, pH 3.5. The sterile-filtered cell culture supernatants were applied directly without any adjustments onto a pre-equilibrated MabSelect Sure column. The column was washed with 10 column volumes of equilibration buffer and the protein eluted isocratically in approx. 1.5 column volumes of elution buffer. Based on UV280 monitoring, pools of fractions containing the eluted proteins were prepared and analyzed using SDS-PAGE/Coomassie, high-pressure liquid chromatography and matrix-assisted laser desorption/ionization time-of-flight mass spectrometry (MALDI-TOF MS) analyses. Each of the two preparations displayed a homogenous and pure protein composition with single dominating molecular weights of approx. 148.1 and 154.8 kDa, respectively. These masses corresponded to the expected values of the two recombinant mAb constructs EP-FpA-PB1.3 and PB1.3. The final protein concentrations were measured using a NanoDrop spectrophotometer (Thermo Scientific, cat. no. ND-1000) together with extinction coefficients of 1.4.

### Example 3

### Thrombin Digests of EP-FpA-PB1.3 and PB1.3 mAb Constructs

To demonstrate thrombin cleavability/activability of the EP-FpA-PB1.3 mAb construct, the protein was digested with plasma-derived human Thrombin (Roche cat. no. 10 602 400 001) prior to a CD62P/P-Selectin-based platelet binding assay. The PB1.3 mAb construct was included as a control. The Thrombin digests were performed by diluting the EP-FpA-PB1.3 and PB1.3 mAb constructs to 0.1 μM in a dilution buffer composed of 50 mM Tris pH 8.2, 150 mM NaCl. Thrombin was dissolved in the same buffer. Thrombin cleavage was tested at 10×-lower, equimolar and 4×-higher molar concentrations compared to the EP-FpA-PB1.3 and PB1.3 mAb constructs. The reactions were followed using SDS-PAGE and MALDI-TOF MS analyses. Control reactions were included in which only buffer and no Thrombin was added to reaction mixtures. A mass reduction of 6.1 kDa for the EP-FpA-PB1.3 mAb construct was observed for all reaction mixtures with Thrombin. When no Thrombin was added, no change in mass was observed for the EP-FpA-PB1.3 mAb construct. The PB1.3 mAb construct which was constructed without an extended N-terminal did not show any change in mass irrespective of Thrombin being present or not in the reaction mixture. The following four samples were prepared for the CD62P/P-Selectin-based platelet binding assay:
Sample 1) EP-FpA-PB1.3 mAb incubated with Thrombin.
Sample 2) EP-FpA-PB1.3 mAb incubated without Thrombin.
Sample 3) PB1.3 mAb incubated with Thrombin.
Sample 4) PB1.3 mAb incubated without Thrombin.

### Example 4

### CD62P/P-Selectin-Based Platelet Binding Assay Using Fluorescence-Activated Cell Sorting (FAGS)

To demonstrate that the Thrombin cleaved EP-FpA-PB1.3 mAb bind specifically to activated platelets containing surface expressing CD62P/P-Selectin, a FACS-based cell binding experiment was performed using purified platelets from healthy donors. In short, blood from healthy donors was drawn by venipuncture of the antecubital vein. Blood was drawn in 9-ml tubes with 3.2% Sodium Citrate (Vacuette containers, ref. nr. 455322, 9NC), shaken carefully and immediately used for analysis. Platelet preparations were produced by making a standard Platelet Rich Plasma (PRP). Here, anti-coagulated whole blood was centrifuged (200 g for 15 minutes) without brake. The upper layer containing PRP was harvested and prostaglandin E (ProstaE) (Sigma, cat. no. P5515) was added at a final concentration of 5 μg/ml for inhibition of platelet activation. Platelets were washed and prepared for staining. To prepare a batch of activated platelets, a dual agonistic activation was performed for 10 min. using 62.5 μg/ml PAR-1 (Bachem., cat. no. H-2936, lot. no. 3000205) and 100 ng/ml Convulxin (Pentapharm, cat. no. 404914/119-02). 50-100,000 cells were used per well. A negative isotype matched mAb named _{CTRL}APC was included in the experiment as a control. An anti-CD62P antibody marker conjugated to RPE (Activated Platelet marker, Becton Dickenson, cat. no 348107 Lot. No. 05338) was used to confirm activation and non-activation of the dual agonistic activated and the ProstaE inhibited PRP preparations.

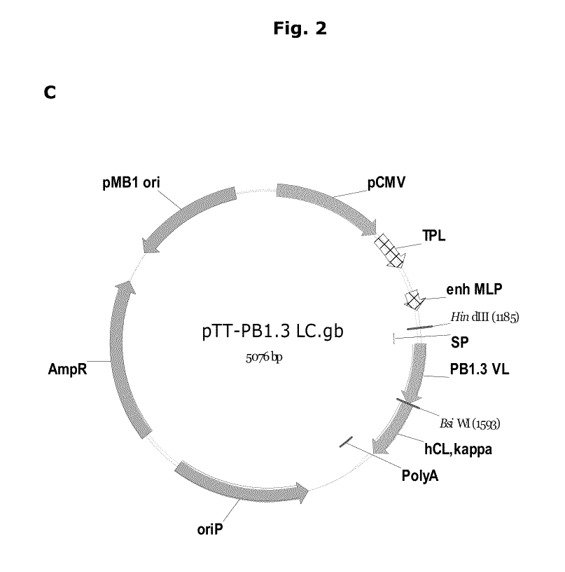

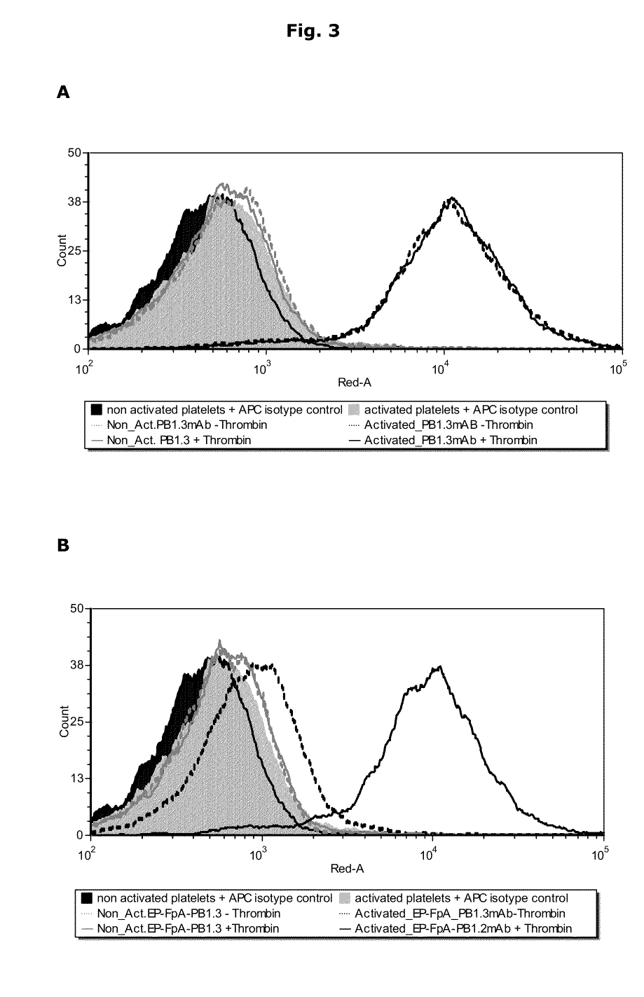

No binding was observed for the PB1.3 mAb construct on non-activated platelets irrespective of pre-incubation with Thrombin (see FIG. 3A). On activated platelets, binding was observed for the PB1.3 mAb construct irrespective of pre-incubation with Thrombin. This confirmed that the PB1.3 mAb binds selectively to activated platelets as described in U.S. Pat. No. 5,800,815. Also, the binding response was similar to the response observed for the commercially available anti-CD62P antibody from Becton Dickenson used as a positive control and marker for platelet activation (data not shown). The EP-FpA-PB1.3 mAb construct displayed no binding to non-activated platelets irrespective of pre-incubation with Thrombin, while binding to activated platelets occurred only for the Thrombin pre-activated sample (FIG. 3B). The isotype matched control antibody _{CTRL}APC did not display any binding to neither non-activated nor activated platelets (FIGS. 3A and 3B). Thus, the shielding effect of the binding domain and Thrombin cleavability/activability of the EP-FpA-PB1.3 was demonstrated in the described cell-based FACS experiment.

### Example 5

### Binding Interaction Analysis

Binding interaction analyses are obtained by Surface Plasmon Resonance in a Biacore T-100 instrument. Capture of the relevant construct at a fixed concentration is obtained by direct immobilization to a CM5 chip of the mAb to a level of 500-1000 RU in 10 mM sodium acetate pH 4.5-5.0. Four-fold dilutions of recombinant human full length CD62P from 200 nM to 0.2 nM are tested for binding to the immobilized construct. Running and dilution buffer: 10 mM HEPES, 150 mM, 0.005% p20, pH 7.4. Regeneration is obtained by 10 mM Glycine, pH 1.7. Determination of kinetic and binding constants (kₒₙ, k_{off}, K_{D}) are obtained assuming a 1:1 interaction of CD62P and the construct of interest using Biacore T100 evaluation software.

Competition of different constructs for binding to CD62P when bound to a given construct is obtained by immobilisation of the given construct to 5000 RU at a CM5 chip, followed by binding of 50 nM CD62P, followed by varying concentrations of the alternative constructs to be tested for competition. Regeneration of the chip is obtained using 10 mM Glycine, pH 1.7.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1-15. (canceled)

16. A construct comprising (i) a monoclonal antibody, or a fragment thereof, (ii) a cleavable motif and (iii) a polypeptide sequence that comprises the epitope motif of (i).

17. The construct of claim 16, further comprising tissue factor or a biologically functional variant or fragment thereof.

18. The construct of claim 16, wherein said monoclonal antibody of said construct is capable of binding a receptor on cell selected from the group consisting of an activated platelet, an activated endothelial cell and a leukocyte.

19. The construct of claim 16, wherein said monoclonal antibody of said construct is capable of binding a receptor on an activated platelet.

20. The construct of claim 19, wherein said receptor is CD62P.

21. The construct of claim 16, wherein said cleavable motif is the cleavage site of a protease.

22. The construct of claim 21, wherein said cleavable motif is fibrinopeptide A (FpA), or a fragment thereof, such as the amino acid sequence corresponding to residues 40-49 (DFLAEGGGVR) of SEQ ID NO: 3.

23. The construct of claim 16, wherein said epitope motif comprises an amino acid sequence corresponding to residues 20-39 (SVLQCLATGNWNSVPPECQA) of SEQ ID NO: 3.

24. The construct of claim 16, wherein said monoclonal antibody, or fragment thereof, can bind its target when said cleavable motif is cloven and can be measured using a fluorescence-activated cell sorting (FACS) analysis.

25. The construct of claim 16, wherein said monoclonal antibody comprises SEQ ID NO: 8.

26. The construct of claim 16, wherein said monoclonal antibody is comprises SEQ ID NO: 9.

27. A polynucleotide that encodes the construct according to claim 16.

28. An isolated cell that is capable of expressing the construct according to claim 16.

29. A method of treating a co-morbidity associated with disruption of a blood vessel comprising administering to a subject in need thereof a construct according to claim 16.


SEQUENCE LISTING (13 sequences)

SEQ ID NO: 1 - protein, 829 aa, Homo sapiens
MANCQIAILYQRFQRVVFGISQLLCFSALISELTNQKEVAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 2 - DNA, 537 bp, Artificial
  EP-FpA-PB1.3 HC-V
aagcttgccgccaccatgggatggtctggtgtatttctttttctcctgagtggtacagctggcgtccactcaagcgtcctgcagtgtctggctacagggaattggaactcagtgcctcctgaatgtcaagccgacttccttgctgaagggggtggtgtgcgcatcgtgggagaggctcaactgcaacagtctgggcccgaactggtagagcctggggcaagtgtgaaggtatcctgcaaagcgtcagggtatacatttactaactacgtgatgcactgggtgaaacagaagccaggacaaggtttggaatggatcgggttcattaatccttccaatgatgggcctaagtataacgaacgattcaagaacaaggctacactgacgtccgataaatcctcctctacagcatacatggaactgagctccttgacttcagaggtaagcgccgtgtacttctgcgcaagagccaggcccggattcgactggtatttcgacgtgtggggcgctggcactaccgtcacagtcagttctgctagc

SEQ ID NO: 3 - protein, 172 aa, Artificial
  EP-FpA-PB1.3 HC-V
MGWSGVFLFLLSGTAGVHSSVLQCLATGNWNSVPPECQADFLAEGGGVRIVGEAQLQQSGPELVEPGASVKVSCKASGYTFTNYVMHWVKQKPGQGLEWIGFINPSNDGPKYNERFKNKATLTSDKSSSTAYMELSSLTSEVSAVYFCARARPGFDWYFDVWGAGTTVTVSS

SEQ ID NO: 4 - DNA, 438 bp, Artificial
  PB1.3 HC-V
aagcttgccgccaccatgggatggtcaggagtttttctttttctgctctctggtactgctggcgtgcatagtgaggctcagcttcagcagtccgggccagaactcgttgaacccggggcttcagtcaaggttagttgcaaggcttcaggttatacattcacaaactacgtaatgcactgggtcaagcagaagccagggcaagggcttgagtggatcgggttcattaatccgtccaatgatggacctaagtacaacgaaaggtttaagaacaaggcaactttgacgagcgacaagagttcctccactgcatatatggagctcagctctctgaccagcgaagttagtgcggtctacttctgcgctagagctaggcctgggttcgattggtactttgatgtgtggggagctggaacaacagttaccgtttcttctgctagc

SEQ ID NO: 5 - protein, 139 aa, Artificial
  PB1.3 HC-V
MGWSGVFLFLLSGTAGVHSEAQLQQSGPELVEPGASVKVSCKASGYTFTNYVMHWVKQKPGQGLEWIGFINPSNDGPKYNERFKNKATLTSDKSSSTAYMELSSLTSEVSAVYFCARARPGFDWYFDVWGAGTTVTVSS

SEQ ID NO: 6 - DNA, 414 bp, Artificial
  PB1.3 LC-V
aagcttgccgccaccatgggcataaagatggaatcacagatacaggtgttcgtgtatatgctgctctggctgtctggcgtggacggcgacatagtgatgacgcagtcacagaaattcatgtccacaagcgtcggggatagggtgagcgttacgtgtaaagcatcccagaacgtagccacaaacgtcgtgtggtatcagcagaggcccggacagtcacccaaggccctcatctataccgcgtcctataggtttagcggtgtgccggagcgcttttcagggtccggaagtggaacagattttactctgacaatcaccaacgtgcaaagtgaggacctcgccgactacttttgccagcaatacaataactatccctatacttttggcggaggcacaaaagtcgaaattcaacgtacg

SEQ ID NO: 7 - protein, 132 aa, Artificial
  PB1.3 LC-V
MGIKMESQIQVFVYMLLWLSGVDGDIVMTQSQKFMSTSVGDRVSVTCKASQNVATNVVWYQQRPGQSPKALIYTASYRFSGVPERFSGSGSGTDFTLTITNVQSEDLADYFCQQYNNYPYTFGGGTKVEIQR

SEQ ID NO: 8 - protein, 447 aa, Artificial
  Heavy chain of anti-CD62P mAB
EAQLQQSGPELVEPGASVKVSCKASGYTFTNYVMHWVKQKPGQGLEWIGFINPSNDGPKYNERFKNKATLTSDKSSSTAYMELSSLTSEVSAVYFCARARPGFDWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 214 aa, Artificial
  Light chain of anti-CD62P mAB
DIVMTQSQKFMSTSVGDRVSVTCKASQNVATNVVWYQQRPGQSPKALIYTASYRFSGVPERFSGSGSGTDFTLTITNVQSEDLADYFCQQYNNYPYTFGGGTKVEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 885 bp, Homo sapiens
atggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttca

SEQ ID NO: 11 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 12 - DNA, 657 bp, Homo sapiens
tcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaa

SEQ ID NO: 13 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE